(19) United States
(12) Patent Application Publication
(10) Pub. No.: US 2019/0345443 A1
(43) Pub. Date: Nov. 14, 2019

(54) METHOD FOR THE CULTURING AND DIFFERENTIATION OF CELLS

(71) Applicants: FRAUNHOFER-GESELLSCHAFT ZUR FORDERUNG DER ANGEWANDTEN FORSCHUNG E.V., Munchen (DE); JULIUS-MAXIMILIANS-UNIVERSITAT WURZBURG, Wurzburg (DE)

(72) Inventors: Martin EMMERT, Munchen (DE); Ferdinand SOMOROWSKY, Wurzburg (DE); Doris HEINRICH, Wurzburg (DE)

(73) Assignees: FRAUNHOFER-GESELLSCHAFT ZUR FORDERUNG DER ANGEWANDTEN FORSCHUNG E.V., Munchen (DE); JULIUS-MAXIMILIANS-UNIVERSITAT WURZBURG, Wurzburg (DE)

(21) Appl. No.: 16/391,889

(22) Filed: Apr. 23, 2019

(30) Foreign Application Priority Data
Apr. 24, 2018 (DE) 10 2018 206 268.4

(51) Int. Cl. C12N 5/0797

(52) U.S. Cl. CPC C12N 5/0623; C12N 2539/00; C12N 2533/12

(57) ABSTRACT

The present invention relates to a method for the culturing of cells on a cell culture substrate, wherein the cell culture substrate comprises a cell culture substrate made of glass and at least a part of the cell culture substrate made of glass has a surface with a nanoporous structure with an average pore diameter of 2 to 150 nm, and the use of a cell culture substrate for the culturing or differentiation of cells, as bottom of a cell culture vessel or bioreactor, as removable insert for cell culture vessels or bioreactors and/or as perfusive membrane for 3D cell culture reactors, whereby the cell culture substrate comprises a cell culture substrate made of glass and at least a part of the cell culture substrate made of glass has a surface with a nanoporous structure with an average pore diameter of 2 to 150 nm.

The present invention relates to a method for the culturing of cells on a cell culture substrate, wherein the cell culture substrate comprises a cell culture substrate made of glass and at least a part of the cell culture substrate made of glass has a surface with a nanoporous structure with an average pore diameter of 2 to 150 nm, and to the use of a cell culture substrate for the culturing or differentiation of cells, as bottom of a cell culture vessel or bioreactor, as removable insert for cell culture vessels or bioreactors and/or as perfusive membrane for 3D cell culture reactors, wherein the cell culture substrate comprises a cell culture substrate made of glass and at least a part of the cell culture substrate made of glass has a surface with a nanoporous structure with an average pore diameter of 2 to 150 nm.

The behavior of viable cells in the complex three-dimensional environments of tissues and organs differs strongly from the behavior of cells on conventional two-dimensional culture surfaces made of polystyrene or silicate-based glasses, which are the standard that is used as culture surfaces for in vitro investigations in the medical device and pharmaceutical industry. Because of this difference in the behavior of the cells to be analyzed, experimental results obtained with common two-dimensional cell culture systems can be applied to the living organism only to a limited extent. Therefore, the close-to-realistic simulation of the physiological conditions in the human or animal body is a particular challenge in the culturing of cells.

In order to attain a close-to-realistic simulation of the cell environment extant in tissues and organs of the human or animal body, inter alia three-dimensional cell culture systems based on matrigel or spheroids are used in the prior art. The three-dimensional cell culturing systems obtained/produced by biological means that are used in this context have crucial disadvantages as compared to conventional two-dimensional cell culture systems, in particular with regard to the utilization in major high-throughput studies. Accordingly, the biological production of the three-dimensional cell culturing systems leads to undesired variations. Moreover, the culturing of cells in three-dimensional cell culture systems is associated with a significantly larger amount of work and considerably higher costs. Further disadvantages include the limited storage capacity and storage stability of the three-dimensional biological cell culture systems available hitherto and the fact that cell cultures of this type are difficult to be viewed under the microscope. In contrast, conventional two-dimensional cell culture systems are known to be in particular characterized by a standardized easy handling, by the ability of these systems to be autoclaved and/or sterilized, by a homogeneous cell colonization due to the planar culture surface, can easily be examined under the microscope, and by the available option of preproduction and storage of such systems at large scale.

One option for attaining a close-to-physiological cell behavior in two-dimensional cell culture systems is to add cytokines or other additives to the culture medium in order to for example induce the migration of the cells or to initiate their differentiation in a certain direction. Due to the use of specifically adapted culture media, this method leads to significant costs and is further disadvantageous in that the differentiation of the cells in standard vessels takes place without any topographic stimulus due to the surface being smooth, wherein the behavior of a cell culture of this type can therefore be applied to the behavior of cells in the human or animal body to a limited extent only.

In order to ensure a close-to-realistic adhesion behavior of cells in two-dimensional systems, the prior art utilizes cell culture vessels that have been made hydrophilic by means of plasma or corona treatments to improve the adhesion of proteins to the surface, or vessels whose surface has been coated directly for this purpose with a cell adhesion-mediating protein, such as fibronectin, vitronectin or poly-L-lysine. However, cell culture vessels coated as described are disadvantageous, because their storage stability is very limited.

Because of the dilemma between easy handling of two-dimensional cell culture systems on the one hand and the physiological cell behavior in three-dimensional cell culture systems on the other hand, there is a strong need for systems that combine the advantages of two-dimensional cell culture systems with a close-to-physiological cell behavior and which can advantageously be integrated into existing standard laboratory devices and high throughput processes.

Therefore, the technical problem underlying the present invention is to overcome the above-mentioned disadvantages of the prior art, in particular by providing a method for the culturing and/or for differentiation of cells, in particular stem cells, wherein the method allows for easy handling of the cell culture and, concurrently, a close-to-realistic simulation of physiological cell behavior.

The present invention solves its underlying problem in particular by the technical teaching of the independent claims.

In this context, the present invention relates to a method for the culturing of cells, comprising the steps of:

a) providing at least one cell that is present in a cell culture medium, and one cell culture substrate;
b) contacting the at least one cell that is present in the cell culture medium with the cell culture substrate;
c) incubating the at least one cell that is present in the cell culture medium on the cell culture substrate;

characterized in that the cell culture substrate comprises a cell culture substrate made of glass and at least a part of the cell culture substrate made of glass has a surface with a nanoporous structure with an average pore diameter of 2 to 150 nm.

Particularly advantageously, the method for the culturing of cells according to the present invention allows the advantages of two-dimensional cell culture systems to be combined with those of three-dimensional cell culture systems and thus in particular allows a close-to-realistic simulation of the physiological behavior of cells to be combined with easy handling. In this context, particularly the use of a cell culture substrate that comprises a cell culture substrate made of glass, wherein at least a part of the cell culture substrate made of glass has a surface with a nanoporous structure with an average pore diameter of 2 to 150 nm, leads to a topographic stimulation of the cells and concurrently allows for the utilization of the work steps and media of conventional two-dimensional cultures. Accordingly, the cell culture substrate comprising a cell culture substrate made of glass as used according to the invention has at least in a part of the surface of the cell culture substrate made of glass, an intrinsic nano-structuring such that in contrast to the known cell culture substrates according to the prior art no subsequent active structuring needs to take place in order to obtain a surface with a nanoporous structure, which inter alia leads to a considerable reduction of the production costs. Moreover, by using the method according to the invention it is advantageously feasible to specifically support and control certain cell functions of different cell types by culturing cells that are present in a cell culture medium on a cell culture substrate made of glass, wherein at least a part of the cell culture substrate made of glass has a surface with a nanoporous structure with an average pore diameter of 2 to 150 nm and through suitable selection of a defined average pore diameter in the range of 2 to 150 nm.

In a further preferred embodiment of the present invention, the at least one cell present in a cell culture medium provided in step a) is a stem cell and the method for the culturing of cells is a method for the differentiation of stem cells. By providing at least one stem cell that is present in a cell culture medium in step a), contacting the at least one stem cell that is present in a cell culture medium with the cell culture substrate in step b), and incubating the at least one stem cell that is present in a cell culture medium on the cell culture substrate in step c), wherein the cell culture substrate comprises a cell culture substrate made of glass and at least a part of the cell culture substrate made of glass has a surface with a nanoporous structure with an average pore diameter of 2 to 150 nm, it is advantageously feasible to initiate a differentiation of the stem cells without the addition of additives. In this context, the surface with a nanoporous structure with an average pore diameter of 2 to 150 nm of the cell culture substrate made of glass acts as a topographic stimulus that initiates the differentiation of the cells.

In a particularly preferred embodiment, the method according to the present invention consists of procedural steps a) to c), i.e. no further procedural steps take place before, after and/or between procedural steps a), b), and c). In a preferred embodiment, the method is implemented in the order of procedural steps a), b), and c).

In a preferred embodiment of the present invention, the cell culture substrate consists completely of glass, wherein the glass has a surface with a nanoporous structure with an average pore diameter of 2 to 150 nm. In a further preferred embodiment of the present invention, the cell culture substrate consists completely of glass, wherein at least a part of the cell culture substrate made of glass has a surface with a nanoporous structure with an average pore diameter of 2 to 150 nm.

In a preferred embodiment of the present invention, the cell culture substrate consists of glass to an amount of at least 20%, preferably at least 25%, preferably at least 30%, preferably at least 35%, preferably at least 40%, preferably at least 45%, preferably at least 55%, preferably at least 60%, preferably at least 65%, preferably at least 70%, preferably at least 75%, preferably at least 80%, preferably at least 85%, preferably at least 90%, preferably at least 95%, preferably at least 98%.

According to the invention, at least a part of the cell culture substrate made of glass, preferably at least 0.1%, preferably at least 0.5%, preferably at least 1%, preferably at least 2%, preferably at least 3%, preferably at least 4%, preferably at least 5%, preferably at least 10%, preferably at least 15%, preferably at least 20%, preferably at least 25%, preferably at least 30%, preferably at least 35%, preferably at least 40%, preferably at least 45%, preferably at least 55%, preferably at least 60%, preferably at least 65%, preferably at least 70%, preferably at least 75%, preferably at least 80%, preferably at least 85%, preferably at least 90%, preferably at least 95%, preferably at least 96%, preferably at least 97%, preferably at least 98%, preferably at least 99%, preferably 100% of the cell culture substrate made of glass has a surface with a nanoporous structure with an average pore diameter of 2 to 150 nm.

In a further preferred embodiment of the present invention, the surface with a nanoporous structure with an average pore diameter of 2 to 150 nm is formed on the cell culture substrate made of glass as an array, preferably as a micro-array. Preferably, an array of this type, preferably a micro-array, is formed of circular or rectangular, in particular square, areas comprising a surface with a nanoporous structure with an average pore diameter of 2 to 150 nm that are arranged on the cell culture substrate made of glass in a preferably regular distance from each other.

In a preferred embodiment of the present invention, the surface with a nanoporous structure of the cell culture substrate, in particular of the cell culture substrate made of glass, has an average pore diameter of 3 to 150 nm, preferably 4 to 150 nm, preferably 5 to 150 nm, preferably 10 to 150 nm, preferably 20 to 150 nm, preferably 30 to 150 nm, preferably 40 to 150 nm, preferably 50 to 150 nm, preferably 60 to 150 nm, preferably 70 to 150 nm, preferably 80 to 150 nm.

It is particularly preferred for the surface with a nanoporous structure of the cell culture substrate, in particular of the cell culture substrate made of glass, to have an average pore diameter of 60 to 140 nm, preferably 70 to 135 nm, preferably 75 to 130 nm, preferably 80 to 125 nm.

In a preferred embodiment of the method for the differentiation of cells, in particular stem cells, according to the present invention, no additives, in particular no cytokines, are added to the cell culture medium.

In a further preferred embodiment of the method for the differentiation of cells, in particular stem cells, according to the present invention, additives, such as cytokines, are added to the cell culture medium. According to said preferred embodiment, it is advantageously feasible to attain the differentiation of cells, in particular stem cells, at a reduced concentration of additives, such as cytokines, as compared to the methods for the differentiation of cells, in particular stem cells, known from the prior art. Moreover, according to said preferred embodiment, it is advantageously feasible to attain an accelerated differentiation of cells, in particular stem cells, as compared to methods for the differentiation of cells, in particular stem cells, known from the prior art.

In a preferred embodiment of the method for the culturing of cells, in particular of the method for the differentiation of stem cells, the cell culture substrate, in particular the cell culture substrate made of glass, has a thickness of 10 to 5000 μm, preferably 20 to 5000 μm, preferably 30 to 4500 μm, preferably 40 to 4000 μm, preferably 50 to 4000 μm, preferably 60 to 3500 μm, preferably 70 to 3000 μm, preferably 80 to 3000 μm, preferably 90 to 2500 μm, preferably 100 to 2000 μm, preferably 150 to 2000 μm, preferably 200 to 1500 μm, preferably 220 to 1000 μm, preferably 240 to 980 μm, preferably 260 to 960 μm, preferably 280 to 940 μm, preferably 300 to 920 μm, preferably 320 to 900 μm, preferably 340 to 880 μm, preferably 360 to 860 μm, preferably 380 to 840 μm, preferably 400 to 820 μm, preferably 420 to 800 μm, preferably 440 to 780 μm, preferably 460 to 760 μm, preferably 480 to 740 μm, preferably 500 to 720 μm, preferably 500 to 700 μm. Preferably, the cell culture substrate, in particular the cell culture substrate made of glass, is a membrane.

In a preferred embodiment of the present invention, the cell culture substrate, in particular the cell culture substrate made of glass, is a porous glass, preferably VYCOR glass. Particularly preferably, the cell culture substrate, in particular the cell culture substrate made of glass, is a porous glass, preferably a VYCOR glass produced according to the method described in U.S. Pat. No. 2,106,744. Preferably, the cell culture substrate, in particular the cell culture substrate made of glass, is a porous glass, preferably a VYCOR glass produced by extraction, in particular by leaching, from phase-separated alkali borosilicate glass.

In a further preferred embodiment of the present invention, the cell culture substrate, in particular the cell culture substrate made of glass, is a glass, whose surface with a nanoporous structure is produced from phase-separated alkali borosilicate glass by partial, preferably complete, extraction, in particular by partial, preferably complete, leaching. The partial extraction, in particular partial leaching, from phase-separated alkali borosilicate glass allows a cell culture substrate, in particular a cell culture substrate made of glass, to be obtained, in which only the surface of the glass has a nano-structuring with an average pore diameter of 2 to 150 nm.

In a preferred embodiment of the present invention, the cell culture substrate, in particular the cell culture substrate made of glass consists of 30 to 80 wt. % silicon dioxide (SiO₂), 20 to 70 wt. % boron oxide (B₂O₃), and 5 to 20 wt. % sodium oxide (Na₂O), preferably of 70 wt. % SiO₂, 23 wt. % B₂O₃, and 7 wt. % Na₂O, before the partial or complete leaching.

In a further preferred embodiment of the present invention, the cell culture substrate, in particular the cell culture substrate made of glass, consists of 50 to 80 wt. % silicon dioxide (SiO₂), 20 to 45 wt. % boron oxide (B₂O₃), and 5 to 20 wt. % sodium oxide (Na₂O) before the partial or complete leaching.

In a preferred embodiment of the present invention, the cell culture substrate, in particular the cell culture substrate made of glass consists of 95 to 98 wt. % SiO₂, 2.5 to 3.5 wt. % B₂O₃, and 0.3 to 0.6 wt. % Na₂O, in particular after partial or complete leaching. Preferably, the cell culture substrate, in particular the cell culture substrate made of glass, comprises at least 95 wt. % SiO₂, preferably at least 95.5 wt. % SiO₂, preferably at least 96 wt. % SiO₂, after the partial or complete leaching.

In a preferred embodiment of the present invention, the cell culture substrate, in particular the cell culture substrate made of glass, has a porosity of 20 to 70%, preferably 21 to 68%, preferably 21 to 66%, preferably 22 to 64%, preferably 22 to 62%, preferably 23 to 60%, preferably 23 to 58%, preferably 24 to 56%, preferably 24 to 54%, preferably 25 to 52%, preferably 25 to 50%, preferably 25 to 48%, preferably 26 to 46%, preferably 26 to 44%, preferably 27 to 43%, preferably 28 to 42%, preferably 29 to 41%, preferably 30 to 40%, preferably 31 to 39%, preferably 32 to 38%, preferably 33 to 37%, preferably 34 to 36%, preferably 35%, in particular after partial or complete leaching.

In a preferred embodiment of the present invention, the surface area of the cell culture substrate made of glass comprising a surface with a nanoporous structure is 10 to 2000 m²/g, preferably 15 to 1500 m²/g, preferably 20 to 1000 m²/g, preferably 20 to 500 m²/g, preferably 50 to 400 m²/g, preferably 60 to 480 m²/g, preferably 70 to 460 m²/g, preferably 80 to 440 m²/g, preferably 90 to 420 m²/g, preferably 100 to 400 m²/g, preferably 100 to 350 m²/g, preferably 100 to 300 m²/g, preferably 120 to 280 m²/g, preferably 140 to 260 m²/g, preferably 160 to 240 m²/g.

In an embodiment of the present invention, the cell culture substrate, in particular the cell culture substrate made of glass, is transparent. In a further preferred embodiment, the cell culture substrate, in particular the cell culture substrate made of glass, is opaque.

In a preferred embodiment of the present invention, the cell culture substrate, in particular the cell culture substrate made of glass, has no oriented surface structure.

In a further preferred embodiment of the present invention, the cell culture substrate, in particular the cell culture substrate made of glass, has no surface coating and/or surface functionalization.

In a further preferred embodiment of the present invention, the cell culture substrate, in particular the cell culture substrate made of glass, has a surface coating and/or surface functionalization.

In a further preferred embodiment of the present invention, the at least one cell is a stem cell, in particular a human stem cell. Preferably, the at least one stem cell is a human mesenchymal stem cell (hMSC), preferably a primary human mesenchymal stem cell. Preferably, the at least one cell, in particular stem cell, is an iPS cell (induced pluripotent stem cell), in particular a human iPS cell (hiPS).

In a further preferred embodiment of the present invention, the at least one cell is a tumor cell. Preferably, the at least one tumor cell is a human tumor cell, preferably a primary human tumor cell.

In a further preferred embodiment of the present invention, the at least one cell is a cell of a tumor cell line. Preferably, the at least one tumor cell is a cell of a human tumor cell line that is well-suited for use in drug tests.

In a further preferred embodiment of the present invention, the at least one cell is a fibroblast. Preferably, the at least one cell is a cell of a human fibroblast cell line that is well-suited for use in standard cytotoxicity tests.

In a preferred embodiment of the present invention, the cell culture substrate is a part of a cell culture vessel or bioreactor, preferably the bottom of a cell culture vessel or bioreactor. In a preferred embodiment of the present invention, the cell culture substrate is a membrane that is applied, preferably welded or sintered, to the bottom of a cell culture vessel or bioreactor. In a further preferred embodiment of the present invention, the cell culture substrate is a membrane that is integrated into the cell culture vessel or bioreactor.

In a preferred embodiment of the present invention, the cell culture substrate is an insert for cell culture vessels or bioreactors, preferably a membrane that can be inserted into the cell culture vessel or into the bioreactor. In this context, the cell culture substrate can be of any shape that is well-suited as an insert for cell culture vessels or bioreactors.

The present invention also relates to the use of a cell culture substrate for the culturing and/or differentiation of cells, wherein the cell culture substrate comprises a cell culture substrate made of glass and at least a part of the cell culture substrate made of glass has a surface with a nanoporous structure with an average pore diameter of 2 to 150 nm.

Moreover, the present invention relates to the use of a cell culture substrate as the bottom of a cell culture vessel or bioreactor, wherein the cell culture substrate comprises a cell culture substrate made of glass and at least a part of the cell culture substrate made of glass has a surface with a nanoporous structure with an average pore diameter of 2 to 150 nm.

Further, the present invention relates to the use of a cell culture substrate as a removable insert of a cell culture vessel or bioreactor, wherein the cell culture substrate comprises a cell culture substrate made of glass and at least a part of the cell culture substrate made of glass has a surface with a nanoporous structure with an average pore diameter of 2 to 150 nm.

The present invention also relates to the use of a cell culture substrate as perfusive membrane for 3D cell culture reactors, wherein the cell culture substrate comprises a cell culture substrate made of glass and at least a part of the cell culture substrate made of glass has a surface with a nanoporous structure with an average pore diameter of 2 to 150 nm.

The embodiments disclosed with reference to the method according to the invention for the culturing of cells shall also apply analogously (mutatis mutandis) to the use of a cell culture substrate made of glass.

In the context of the present invention, the term “cell culture substrate” shall be understood to refer to a material on which a growth of cells can take place. In this context, the “cell culture substrate” according to the present invention comprises a cell culture substrate made of glass, wherein at least a part of said cell culture substrate made of glass has a surface with a nanoporous structure with an average pore diameter of 2 to 150 nm. This means that the term “cell culture substrate” includes embodiments, in which the entire cell culture substrate consists of glass and at least a part of said cell culture substrate made of glass, preferably the entire cell culture substrate made of glass, has a surface with a nanoporous structure with an average pore diameter of 2 to 150 nm. On the other hand, the term also includes embodiments, in which the cell culture substrate according to the present invention consists of various materials, wherein at least a part of the cell culture substrate consists of glass, of which at least a part has a surface with a nanoporous structure with an average pore diameter of 2 to 150 nm. Also conceivable in this context are for example embodiments, in which only certain areas of the cell culture substrate made of glass have a surface with a nanoporous structure with an average pore diameter of 2 to 150 nm and other areas of the cell culture substrate made of glass possess no such surface with a nanoporous structure.

In the context of the present invention, the term “intrinsic nano-structuring” of the cell culture substrate shall be understood to mean that at least a part of the cell culture substrate made of glass has a surface with a nanoporous structure, i.e. a surface with pores with an average pore diameter of 2 to 150 nm, in particular of 3 to 150 nm, preferably 4 to 150 nm, preferably 5 to 150 nm, preferably 10 to 150 nm, preferably 20 to 150 nm, preferably 30 to 150 nm, preferably 40 to 150 nm, preferably 50 to 150 nm, preferably 60 to 150 nm, preferably 70 to 150 nm, preferably 80 to 150 nm.

In the context of the present invention, the term “and/or” shall be understood to mean that all members of a group that are connected by the term “and/or” are disclosed as an alternative to each other as well as cumulative with each other in any combination.

In the context of the present invention, the term “comprising” shall be understood to mean that elements not explicitly specified may be added to the elements explicitly specified by said term. In the context of the present invention, said term shall also be understood to mean that only the explicitly specified elements are included and no further elements are present. In said particular embodiment, the meaning of the term “comprising” is identical to the term “consisting of”. Moreover, the term “comprising” shall also include entireties that contain, aside from the explicitly specified elements, further non-specified elements that are of functionally and qualitatively subordinate or coordinate nature. In said embodiment, the meaning of the term “comprising” is identical to the term “essentially consisting of”.

Further preferred embodiments are evident from the sub-claims.

The present invention shall be illustrated based on the following examples and related figures.

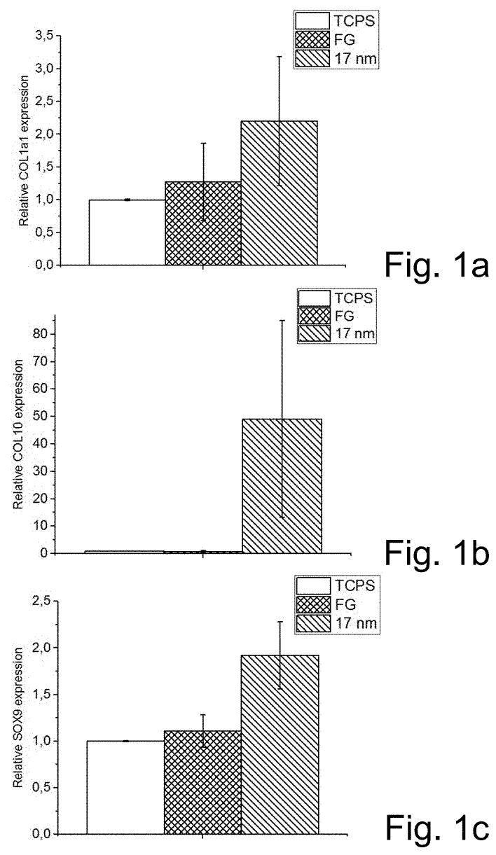

FIG. 1 shows the relative expression of the cartilage-specific genes Col1a1 (FIG. 1a), Col10 (FIG. 1b), and Sox9 (FIG. 1c) in primary human mesenchymal stem cells (hMSCs) of two patients on two control surfaces (TCPS=tissue culture polystyrene, FG=flat cover glass) after 7 to 12 days as compared to the growth of the cells on the cell culture substrate according to the present invention (average pore diameter 17 nm, bars represent the means of the two patients).

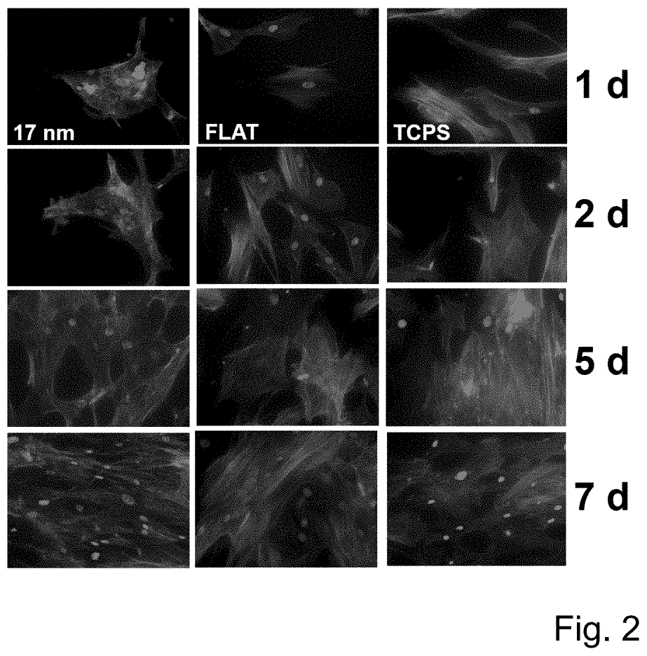

FIG. 2 shows a phalloidin staining of the actin cytoskeleton of primary human mesenchymal stem cells (hMSCs) grown on a nanoporous glass membrane with an average pore diameter of 17 nm (left) and of cells grown on the two control substrates (middle, right) after 1, 2, 5, and 7 days.

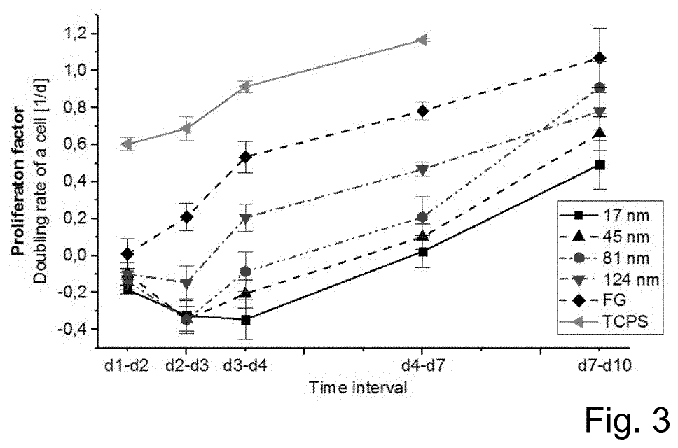

FIG. 3 shows proliferation rates of L929 fibroblasts in defined periods of time on cell culture substrates according to the present invention with different average pore diameters and on two control surfaces, each under standard cell culture conditions (TCPS=tissue culture polystyrene, FG=flat cover glass).

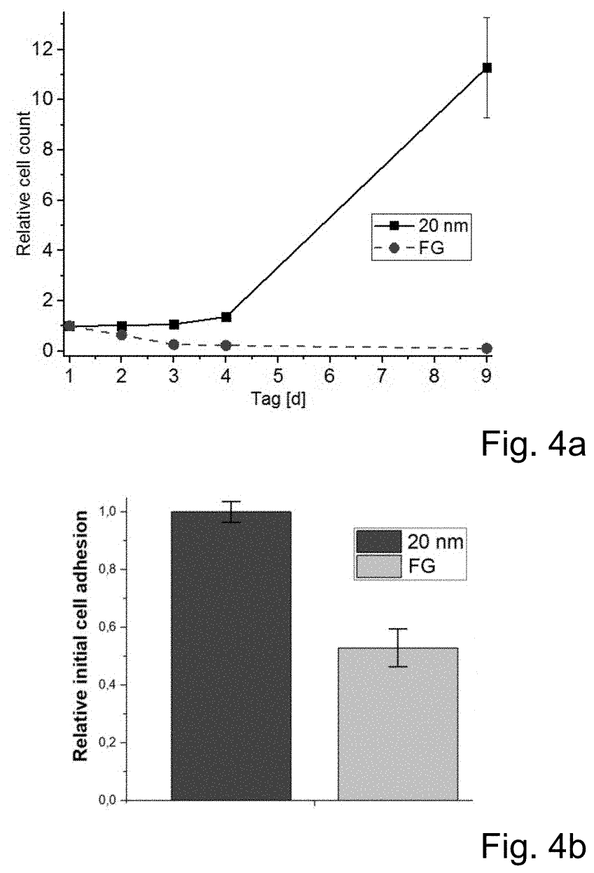

FIG. 4a shows the development of the relative cell count of SK-MEL-28 melanoma cells in overhead culture on cell culture substrates according to the present invention with an average pore diameter of 20 nm and on flat cover glasses (FG).

FIG. 4b shows the adhesion of SK-MEL-28 melanoma cells on a nanoporous glass membrane with an average pore diameter of 20 nm and on a flat non-porous glass surface (FG) after 3 hours of incubation on the respective substrate.

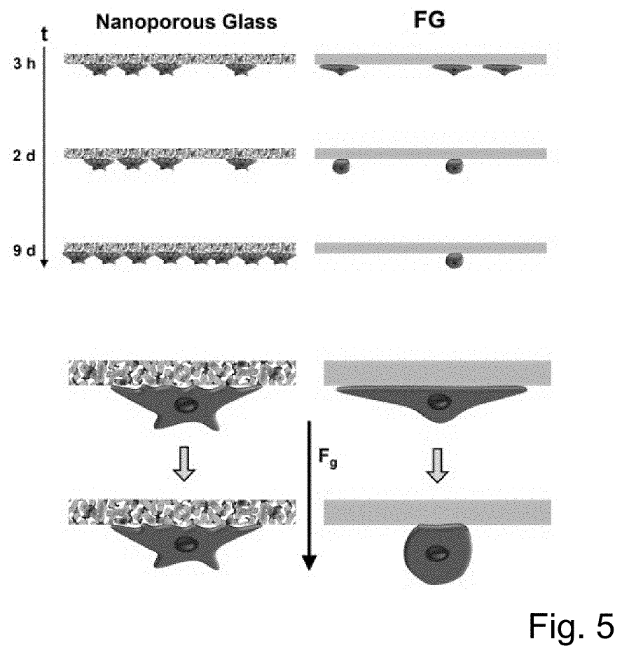

FIG. 5 schematically shows the morphology and adhesion of SK-MEL-28 melanoma cells grown on a nanoporous glass membrane versus SK-MEL-28 melanoma cells grown on a flat non-porous glass surface (FG) in overhead culture at different points in time.

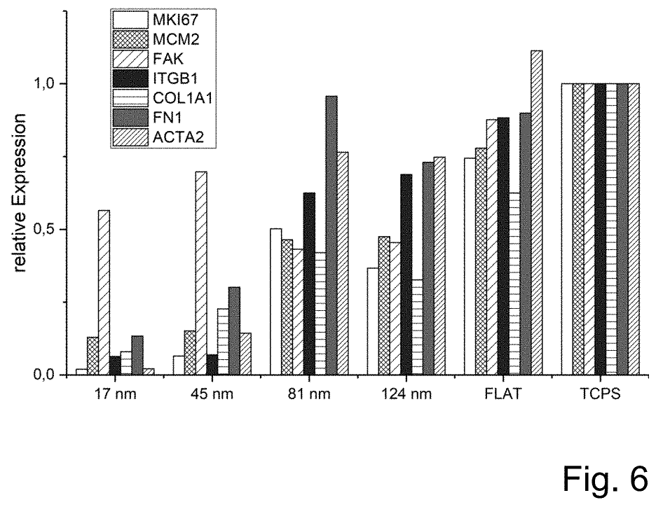

FIG. 6 shows the analysis of the mRNA expression of L929 cells after 48 h of culturing on the different nanoporous glass membranes (17 nm, 45 nm, 81 nm, 124 nm) and on the two control surfaces (FG, TCPS).

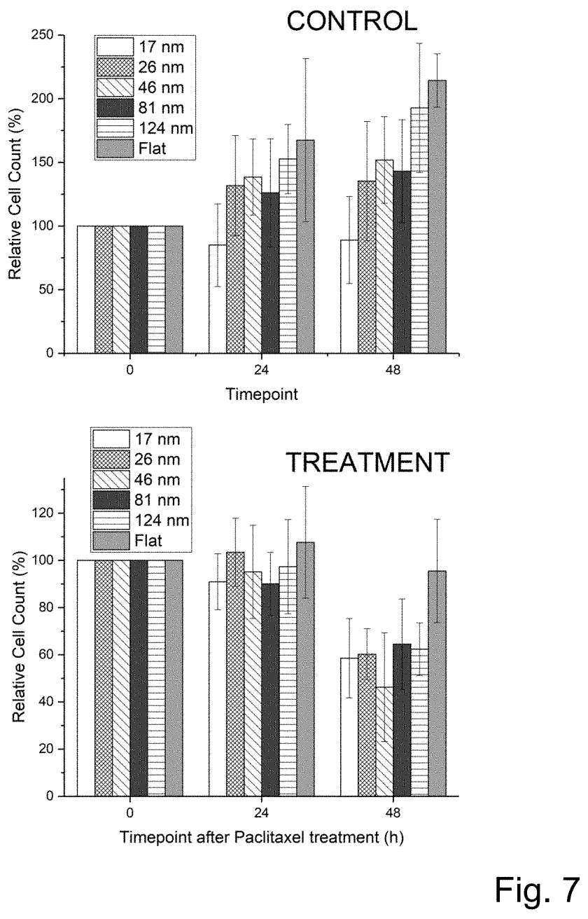

FIG. 7 shows the development of the relative cell count of MDA-MB-231 breast cancer cells in overhead culture on cell culture substrates according to the present invention with different average pore diameters (17 nm, 26 nm, 46 nm, 81 nm, 124 nm) and on flat cover glasses (FG) without active agent (CONTROL) and exposed to 500 nM paclitaxel in each case (TREATMENT).

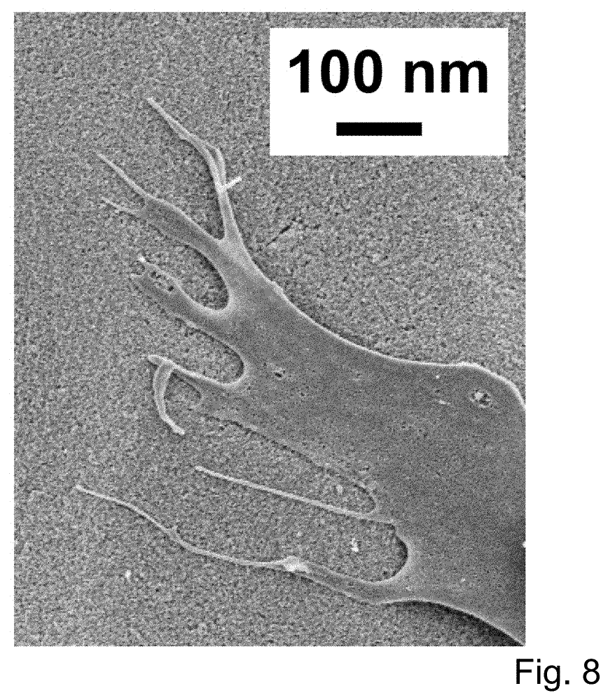

FIG. 8 shows a scanning electron micrograph of a lamellopodium of a human mesenchymal stem cell (hMSC) with many small filopodia after two days of incubation on a cell culture substrate according to the invention with an average pore diameter of 17 nm.

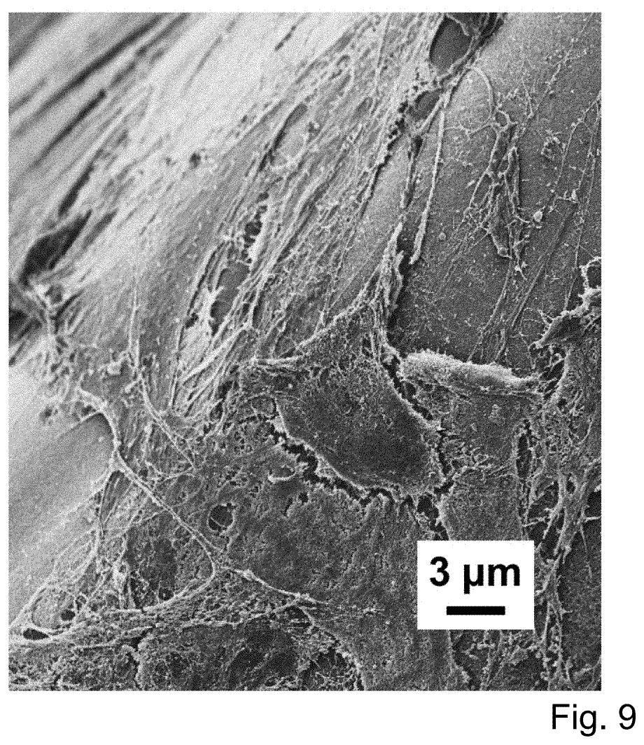

FIG. 9 shows a scanning electron micrograph of human mesenchymal stem cells (hMSCs) incubated for two days on a cell culture substrate according to the present invention with an average pore diameter of 17 nm.

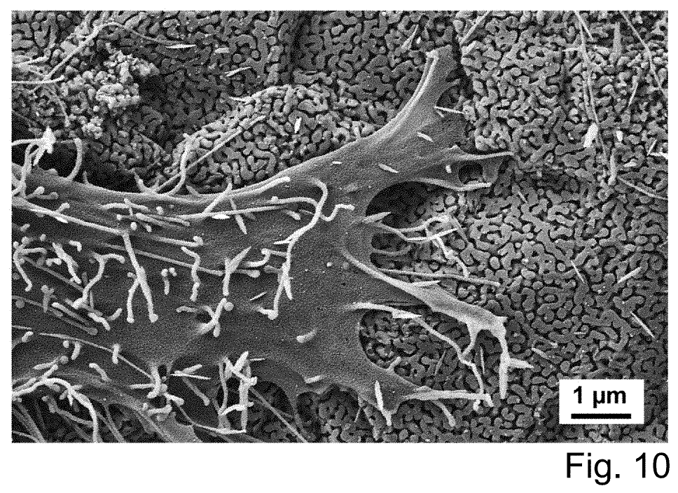

FIG. 10 shows a scanning electron micrograph of a lamellopodium of an L929 fibroblast with many small filopodia after two days of incubation on a cell culture substrate according to the invention with an average pore diameter of 124 nm.

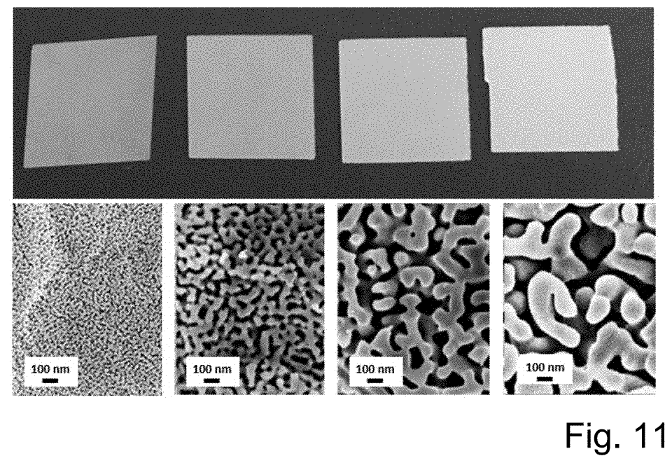

FIG. 11 shows four different nanoporous glass membranes according to the present invention (top) and scanning electron micrographs of the nanoporous surface structure of the individual membranes.

### EXAMPLES

### 1. Production and Physical Properties of Nanoporous Glass Membranes of Different Pore Size

In order to test the influence of nanoporous glass on the behavior of viable cells and the dependence on the pore diameter, a modified VYCOR process was used to produce glass membranes with different average pore diameter for Examples 2 to 7. It was evident that the membranes became increasingly opaque with increasing temperature during leaching, which indicates that the average pore diameter was increased (FIG. 11). This macroscopic observation was confirmed by UV/VIS experiments from which it was clearly evident that increasing temperature during phase separation is associated with a broadening of the range of the wavelengths absorbed by the nanoporous glass. Controlling the temperature during the phase separation, cooling process, and controlled leaching, enables to produce nanoporous glass membranes with an average pore diameter between 17 and 124 nm and a thickness of only 250 μm.

2. Culture and Induction of Chondrogenic Differentiation of hMSCs

In order to test the ability of the cell culture substrates according to the present invention to induce a chondrogenic differentiation, primary hMSCs of two patients were incubated on two control surfaces (TCPS=tissue culture polystyrene, FG=flat cover glass) and on a cell culture substrate according to the present invention, namely a cell culture substrate comprising a VYCOR membrane with a nanoporous structure with an average pore size of 17 nm, and the relative expression of the cartilage-specific genes Col1a1, Col10, and SOX9 was determined by means of qPCR.

Compared to the two control surfaces, a clear increase of the relative expression of Col1a1 (FIG. 1a), Col10 (FIG. 1b), and SOX9 (FIG. 1c) was evident upon incubation of the cells on a cell culture substrate according to the present invention.

In addition, the actin cytoskeleton of cells grown on the nanoporous glass membrane with an average pore diameter of 17 nm and of the cells grown on the two control substrates was stained with phalloidin. It was evident that the actin filaments in the cells cultured on the 2D control surfaces were significantly more well-ordered than the actin filaments of the cells cultured on nanoporous glass membranes (FIG. 2).

Said induction of a chondrogenic differentiation without the addition of external media additives on a cell culture substrate according to the present invention as early as after the first week advantageously allows for the utilization of cell culture substrates according to the present invention as surface for rapid and inexpensive differentiation of hMSCs.

### 3. Comparison of the Proliferation Rates of L929 Fibroblasts on Cell Culture Substrates According to the Present Invention Versus Proliferation Rates on Control Surfaces

The cell proliferation on standard 2D surfaces often differs strongly from the proliferation inside the human body since the cells in the body are situated inside 3D tissues and often proliferate individually, whereas a usually uncontrolled growth of the cells is possible on a standard 2D surface.

In the present experiment, L929 fibroblasts were seeded and incubated under standard 2D culture conditions on two control surfaces (TCPS=tissue culture polystyrene, FG=flat cover glass) and on different cell culture substrates according to the present invention, namely cell culture substrates, each of which having a VYCOR membrane with a nanoporous structure with different average pore diameters (17 nm, 45 nm, 81 nm, 124 nm). After just a few days, the L929 fibroblasts reached similar proliferation rates on the cell culture substrates according to the present invention as on the smooth control surfaces (FIG. 3).

Accordingly, similar proliferation rates as upon the growth of cells on standard 2D surfaces can be attained on the cell culture substrates according to the present invention with topographic stimulation of the cells by the surface with a nanoporous structure.

### 4. Proliferation of SK-MEL-28 Melanoma Cells in Overhead Culture on Cell Culture Substrates According to the Present Invention Versus Proliferation on Smooth Glass Surfaces

For investigation of the proliferation of SK-MEL-28 melanoma cells in overhead culture on the surfaces of the cell culture substrates according to the present invention with a nanoporous structure versus the growth of cells on smooth glass surfaces, SK-MEL-28 melanoma cells were seeded on the different substrates and incubated in overhead culture for a period of 9 days. In this context, the cells that had been incubated on the cell culture substrates according to the present invention (cell culture substrate with nanoporous VYCOR membrane) with an average pore diameter of 20 nm were detected to show strong proliferation in overhead culture, whereas the cell count on the smooth glass surfaces decreases steadily under the same conditions (FIG. 4a).

In particular, it was evident that as early as after 3 hours of incubation on a flat non-porous glass surface, the adhesion of SK-MEL-28 melanoma cells with a relative cell count of 0.53±0.07 was clearly lower than the adhesion of SK-MEL-28 melanoma cells on a nanoporous glass membrane with an average pore diameter of 20 nm (FIG. 4b).

In addition, scanning electron micrographs showed that the cells grown on a flat non-porous glass surface significantly more often comprise a circularity and a higher solidity, which is indicative of a rather passive spreading process with a more circular morphology and fewer filopodia. In contrast thereto, the cells grown on nanoporous glass membranes had more filopodia and occupied a larger area of the substrate surface, which is indicative of an active spreading process with strong focal adhesion of the cells to the topographic surface in overhead culture (FIG. 5).

Thus, the cell culture substrates according to the present invention advantageously allow the cell adhesion to be improved by simulating a three-dimensional environment even under the effect of gravity and without additional functionalization/coating. Accordingly, the surface of the cell culture substrates according to the present invention resembles the natural environment in the human body more closely than smooth 2D surfaces.

5. Different mRNA Expression on Nanoporous Glass Membranes with Different Average Pore Diameter

The mRNA expression of L929 cells on the different nanoporous glass membranes (17 nm, 45 nm, 81 nm, 124 nm) was analyzed by means of qPCR after 48 h of culturing, i.e. during the initial resting phase, in which the cells settle on the surface of the membranes (FIG. 6). It is evident that in particular cells that are being cultured on nanoporous glass membranes with an average pore diameter of 81 nm or 124 nm show an mRNA expression profile that is very similar to the one of cells cultured on a flat non-porous glass surface. This shows a positive interaction between the cells and the surface, although no extensive proliferation of the cells has commenced at this point in time. Moreover, the induction of cell proliferation is significantly increased in the presence of the nanoporous glass membranes with an average pore diameter of 81 nm or 124 nm as compared to the other nanoporous glass membranes. This is evident from the increased expression of proliferation-specific proteins (MKI67, MCM2). In addition, genes regulating other cell functions, such as cell adhesion (FAK, Itgb1), matrix production (COL1A1, FN1), and contraction (ACTA2), were also analyzed. There is a notable reduced expression of ACTA2 by the cells cultured on the nanoporous glass membranes as compared to cells cultured on the flat non-porous glass surface. A drastic change of the expression profile is detectable below an average pore diameter of 80 nm, wherein cells cultured on these nanoporous glass membranes have a clearly increased expression of PTK2/FAK (focal adhesion kinase), whereas other essential genes are strongly down-regulated.

### 6. Simulation of the Physiological Adhesion Mechanism of Cells to Demonstrate the Effectiveness of Cytoskeleton-Effective Agents

In the present experiment, MDA-MB-231 breast cancer cells were initially seeded on cell culture substrates according to the present invention, in particular nanoporous glass membranes with average pore diameters of 17 nm, 26 nm, 46 nm, 81 nm, and 124 nm, and on a smooth non-porous glass surface and cultured for 24 h in order to obtain homogeneous cell colonization on all substrates. Subsequently, the samples were inverted and divided into two groups: one control group and one test group, wherein the culturing took place in overhead culture for 48 h. In this context, the control group was cultured in normal culture medium and 500 nM paclitaxel was added to the culture medium of the test group. During culturing for 48 h in overhead culture, a reduction of the relative cell count on the substrates according to the invention by approximately 35-55% in the test group as compared to the control group was observed (FIG. 7). Interestingly, the reduction of the relative cell count within the 48 h period was considerably lower on the smooth non-porous glass surface (approximately 5%).

The present result shows the feasibility of simulating the physiological adhesion mechanism on the cell culture substrates according to the present invention and indicates the suitability of the cell culture substrates for demonstration of the effectiveness of agents that intervene in cytoskeletal processes.

7. Proliferation of Primary Human Mesenchymal Stem Cells (hMSC) on Nanoporous Glass Membranes of Different Pore Size

Primary hMSC were seeded on nanoporous glass membranes having three different average pore diameters and two control substrates (TCPS=tissue culture polystyrene, FG=flat cover glass). The samples were fixated with glutaraldehyde at different points in time and prepared for scanning electron microscopy. All tested samples showed good cell adhesion and cell proliferation. During the first days of culturing on the nanoporous glass membranes, the formation of cell clumps was observed. These were no longer present after day 3, which indicated full spreading of the cells.

1. A method for the culturing of cells, the method comprising:
a) providing at least one cell that is present in a cell culture medium, and a cell culture substrate;
b) contacting the at least one cell that is present in the cell culture medium with the cell culture substrate;
c) incubating the at least one cell that is present in the cell culture medium on the cell culture substrate;
wherein the cell culture substrate comprises a cell culture substrate made of glass and at least a part of the cell culture substrate made of glass has a surface with a nanoporous structure with an average pore diameter of 2 to 150 nm.

2. The method according to claim 1, wherein the at least one cell that is present in the cell culture medium provided in step a) is a stem cell, and the method is a method for the differentiation of stem cells.

3. The method claim 1, wherein the surface with a nanoporous structure has an average pore diameter of 40 to 150 nm.

4. The method according to claim 1, wherein no additives are added to the cell culture medium.

5. The method according to claim 1, wherein the cell culture substrate has a thickness of 10 to 500 μm.

6. The method according to claim 1, wherein the cell culture substrate is transparent.

7. The method according to claim 1, wherein the cell culture substrate has at least one of a surface functionalization and surface coating.

8. The method according to claim 1, wherein the cell culture substrate is a part of a cell culture vessel or a bioreactor.

9. The method according to claim 1, wherein the cell culture substrate is an insert for cell culture vessels or bioreactors.

10. The method according to claim 1, wherein the cell culture substrate is a removable insert for cell culture vessels or bioreactors.

11. The method according to claim 1, wherein the cell culture substrate is a bottom of a cell culture vessel or bioreactor.

12. The method according to claim 1, wherein the cell culture substrate is a perfusive membrane for 3D cell culture reactors.

13. The method claim 1, wherein the surface with a nanoporous structure has an average pore diameter of 80 to 150 nm.

14. The method according to claim 1, wherein the cell culture substrate made of glass has at least one of a surface functionalization and surface coating.
